(19) United States
(12) Patent Application Publication
(10) Pub. No.: US 2006/0168675 A1
(43) Pub. Date: Jul. 27, 2006

(54) Novel polypetide having function of 7-keto-8-aminopelargonic acid synthase of plant and method for inducing growth inhibition and lethality by suppressing expression of the polypeptide

(75) Inventors: Dong-Hee Lee, Busan (KR); In-Taek Hwang, Taejon (KR); Jung-Sup Choi, Taejon (KR); Yong-Ki Min, Taejon (KR); Tae-Joon Kim, Taejon (KR); Jae-Heung Ko, Seoul (KR); Tae-Hoon Kim, Busan (KR); Yu-Shin Park, Gyeongsangbuk-do (KR); Kwang-Yun Cho, Taejon (KR); Seon-Woo Lee, Taejon (KR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GENOMINE, INC, Pohang (KR)

(21) Appl. No.: 10/519,476

(22) PCT Filed: Jul. 2, 2003
(86) PCT No.: PCT/KR03/01301

§ 371 Date: Dec. 30, 2004

(30) Foreign Application Priority Data
Jul. 2, 2002 (KR) 10 2002 0038011

(51) Int. Cl. A01H 1/00; C07H 21/04; C12N 9/10; C12N 15/82; C12N 5/04

(52) U.S. Cl. 800/278; 435/193; 435/419; 435/468; 536/23.2

(57) ABSTRACT

The invention relates to a novel polypeptide participating in biotin biosynthesis of plant, a polynucleotide encoding the said polypeptide, a method for inducing plant growth inhibition by suppressing the expression or function of the said polypeptide, resulting in inhibition of biotin biosynthesis, and a method for identifying herbicidal compounds that inhibit the expression or function of the said polypeptide. Biotin biosynthesis is an essential process for the growth of plants, however, the process is not present in both human and animals. Therefore, the compounds suppressing the expression or function of the novel polypeptide that relates to biotin biosynthesis can be effectively used as a new herbicide doing human and animals no harm.

### FIELD OF THE INVENTION

The present invention relates to a novel polypeptide isolated from plant and a polynucleotide encoding the same, more particularly a novel polypeptide involved in biotin biosynthesis of plant, a polynucleotide encoding the said polypeptide, a method for inducing plant growth inhibition by suppressing expression or function of the said polypeptide, resulting in inhibition of biotin synthase activity, and a method for identifying herbicidal compounds that inhibit the expression or the function of the said polypeptide.

### BACKGROUND OF THE INVENTION

As the most recently found member of vitamin B group, biotin is essential for the growth of both animals and plants. Biotin plays an important role as a coenzyme in transporting CO₂ to a specific substrate during carboxylation, decarboxylation and transcarboxylation related to fatty acid or carbohydrate metabolism. Bacteria, plants and some of algae can synthesize biotin in vivo, while most algae and animals cannot, suggesting that they should get biotin from outside.

Since the biotin biosynthesis pathway was first disclosed in Escherichia coli and Bacillus subtilis (Eisenberg M A, Adv. Enzymol., 38:317-372, 1973; Pai C H, J. Bacteriol., 121:1-8, 1975), studies have been actively made on the biotin biosynthesis pathway of bacteria based on biochemistry and genetics (Ploux and Marquet, Biochem. J., 283:327-331, 1992; Alexeev et al., J. Mol. Biol., 235:774-776, 1994; Huang et al., Biochemistry, 34:10985-10995, 1995).

As a result, which gene plays what role in each stage of biotin biosynthesis pathway in microorganisms such as Escherichia coli and Bacillus subtilis has been clearly answered. Particularly, it has been confirmed that Escherichia coli has a bio cluster consisting of 5 genes of bioABFCD and Bacillus subtilis has a bio cluster consisting of 6 genes of bioWAFDBI (Bachman B J, Microbiol. Rev., 54:130-197, 1990; Bower et al., J. Bacteriol., 178:4122-4130, 1996). Bacillus sphearicus was confirmed to have two independent clusters, bioXWF and bioDAYB. In addition, it was confirmed that the expression of biotin in Escherichia coli is suppressed by the attachment of BirA protein to the region between bioA and bioB genes (Barker and Campbell, J. Mol. Biol., 146:469-492, 1981).

The first step of biotin biosynthesis in Escherichia coli is decarboxylative condensation catalyzed by KAPA synthase (7-keto-8-aminopelargonic acid synthase), in which L-alanine and pimeloyl CoA were converted into KAPA (7-keto-8-aminopelargonic acid, also known as 8-amino-7-oxononanoate; AON). KAPA synthase, a bioF gene product of Escherichia coli, is a pyridoxal 5′-phosphate-dependent enzyme and has about 42 kDa molecular weight.

As seen hereinbefore, biotin biosynthesis pathway of microorganism has been well known, while that in plants has not been explained yet, especially there are much to be known about KAPA synthase playing a role in the early stage of biotin biosynthesis. However, some interesting evidence has been reported that biotin biosynthesis is occurring in plants following the same pathways as in Escherichia coli Baldet et al., Eur. J. Biochem., 217:479-485, 1993). Moreover, biotin biosynthesis in plants and the use thereof have also been disclosed by the research on biotinylated protein (Nikolau et al., Anal. Biochem., 149:448-453, 1985; Tissot et al., Biochem. J, 314:391-395, 1996), and the isolation and the research on the characteristics of auxotrophic mutants. Particularly, the detailed analysis on mutants like auxotrophic mutants offered great information about biotin biosynthesis and its regulation.

Firstly, converting reaction from KAPA to 7,8-diaminopelargonic acid (DAPA) is absent in biol auxotrophic mutant of Arabidopsis thaliana (Meinke D W, Theor. Appl. Genet., 72:543-552, 1985). And the last step of biotin biosynthesis pathway, which is converting reaction from dethiobiotin to biotin, is absent in bio2 mutant (Patton et al., Plant Physiol., 116:935-946, 1998).

The above studies support the possibility to develop an environmental friendly herbicide based on the study on biotin biosynthesis in plants. Biotin biosynthesis is indispensable procedure for plant growth, but the process is not present in both human and animals. Thus, it could be possible to inhibit the growth of harmful plants by suppressing biotin biosynthesis, without doing harm to human and animals. Rendina et al. tried to develop a new herbicide by targeting bacterial dethiobiotin synthetase (DTBS) (Rendina et al., Pesticide Sci., 55:236-247, 1999). But, as of today, biotin biosynthesis in plants has not been fully explained and the studies on a method to inhibit the growth of harmful plants by targeting genes related to biotin biosynthesis, which seems to be safe and environmental friendly, have just begun. And the functions of metabolism in plants have not been clearly disclosed when the conventional herbicides are used.

### DISCLOSURE OF THE INVENTION

Thus, the present inventors have studied on the biotin biosynthesis pathway taking Arabidopsis thaliana as a major subject. As a result, the inventors isolated a polypeptide participated in the first step of biotin biosynthesis pathway and a polynucleotide encoding the same, and analyzed the functions thereof. And also, the inventors confirmed that the suppression of the expression or function of the said polypeptide could be a fatal blow for plant growth. The present inventors have completed this invention by confirming that the said polynucleotide and the polypeptide expressed therefrom could be excellent candidates for developing novel herbicides.

It is an object of the present invention to provide a method for the development of a novel environmental friendly herbicide inducing plant growth inhibition by suppressing biotin biosynthesis in plants.

In order to achieve the above object, the present invention provides an isolated polypeptide having an amino acid sequence selected from the group consisting of;

(a) an amino acid sequence of SEQ. ID. NO. 2, and

(b) an amino acid sequence having at least 70% identity to SEQ. B. NO. 2.

The present invention also provides an isolated polynucleotide encoding the said polypeptide or an antisense polynucleotide complementary to the same.

The present invention provides an expression vector comprising the isolated polynucleotide encoding the said polypeptide or the antisense polynucleotide complementary to the same.

The present invention provides a cell comprising the said expression vector.

The present invention provides a transgenic plant or seed comprising the said expression vector.

The present invention further provides a method for inducing plant growth inhibition by suppressing the expression or the function of the said polypeptide, resulting in the inhibition of biotin biosynthesis.

The present invention yet further provides a method for identifying herbicidal compounds that inhibit the expression or function of the said polypeptide.

In the present invention, the gene encoding KAPA synthase of Arabidopsis thaliana is stated in italics such as “AtKAPAS polynucleotide”, “AtKAPAS gene” or “AtKAPAS” and the protein encoded thereby is stated as “AtKAPAS polypeptide”, “AtKAPAS protein” or “AtKAPAS”.

Further features of the present invention will appear hereinafter.

The present invention provides a novel polypeptide involved in biotin biosynthesis. The polypeptide has the same function as E. coli KAPA synthase that plays a role in converting L-alanine and pimeloyl CoA into KAPA (7-keto-8-aminopelargonic acid) in the first step of biotin biosynthesis pathway in E. coli.

The range of polypeptide according to the present invention may include a polypeptide having an amino acid sequence represented by SEQ ID No. 2, and functional equivalents thereof. The “functional equivalents” refer to polypeptides having more than 70%, preferably more than 80%, more preferably more than 90% identity (sequence homology) to the amino acid sequence of SEQ ID No. 2 as a result of addition, substitution or deletion of one or more amino acids, and exhibiting substantially the same physiological activity of the polypeptide of SEQ ID No. 2. Also, “substantially the same physiological activity” means an activity converting L-alanine and pimeloyl CoA into KAPA (7-keto-8-aminopelargonic acid) in the first step of biotin biosynthesis pathway. The protein according to the present invention can be obtained from nature (for example, plant cells) or by expression of a recombinant nucleic acid that encodes the protein or by a chemical synthesis. Preferably, the protein can be isolated from Arabidopsis thaliana.

The present invention also provides a polynucleotide encoding the said polypeptide. More particularly, the present invention provides an AtKAPAS (Arabidopsis thaliana KAPA synthase), a polynucleotide having a nucleotide sequence represented by SEQ. ID. NO. 1. The AtKAPAS has about 51.3 kDa molecular weight and comprises 1410 bp size open reading frame encoding 469 amino acids.

The present invention provides an antisense polynucleotide complementary to the said polynucleotide. More particularly, the present invention provides an antisense polynucleotide complementary to the polynucleotide having a nucleotide sequence of SEQ. ID. NO. 1. Antisense polynucleotides are generally known to combine with target nucleotides in nucleic acid (RNA or DNA), by which they suppress the synthesis or function of the nucleic acid. Particularly, an antisense polynucleotide corresponding to a specific gene can be hybridized with both RNA and DNA, leading to the suppression of the expression of the specific gene in the level of transcription or translation.

Meanwhile, the amino acid sequence encoded by the AtKAPAS shows 28%, 34% and 38% homology and 43%, 52% and 54% similarity, respectively, to the sequences of bioF genes (GeneBank accession number NP286539, NP390900 and JQ0512, each) encoding KAPA synthase, a protein involved in biotin biosynthesis of Escherichia coli, Bacillus subtilis and Bacillis sphaericus. The amino acid sequence also comprises aminotransferase class I and class II domain at its C-terminal, suggesting that the polynucleotide of the invention has the function of aminotransferase in addition to that of KAPA synthase. Besides, the amino acid sequence comprises a domain presumed to be plasma membrane spanning region.

The polynucleotide of the present invention that encodes the polypeptide involved in biotin synthesis or the antisense polynucleotide that is complementary thereto can be inserted to a suitable expression vector to transform plant cells. The “expression vector” refers to a known plasmid, virus or other medium into which the polynucleotide sequence encoding the polypeptide involved in biotin synthesis or the antisense polynulceotide complementary thereto can be inserted or introduced. The gene sequence of the present invention can be operably linked to the expression control sequence. The operably linked gene sequence and expression control sequence can be included within a single expression vector containing a selective marker and a replication origin. The “operably linked gene sequence and expression control sequence” may be a gene sequence and an expression control sequence which are linked in such a manner to enable the expression of the gene when a suitable molecule is bound to the expression control sequence. The “expression control sequence” means a DNA sequence controlling the expression of a nucleic acid sequence which is operably linked in a particular host cell. Such a control sequence includes a promoter for performing transcription, an operator sequence for controlling transcription, a sequence encoding a suitable mRNA ribosome binding site, and a sequence controlling termination of transcription or translation. A suitable vector into which the gene of the present invention can be introduced is a Ti plasmid, a root inducing (Ri) plasmid or a plant virus vector. The suitable vector may be, but not limited to, a binary vector of pCAL-n or pCAMBIA series. Anyone skilled in the pertinent art can select a suitable vector for introducing the nucleic acid of the gene of the present invention. Any vector capable of introducing the polynucleotide sequence encoding the polypeptide involved in biotin synthesis or the antisense polynulceotide complementary thereto into plant cells can be used in the present invention.

The expression vector according to the present invention can be introduced into cells by known methods. The cells may be eukaryotic cells such as yeast or plant cell, or prokaryotic cells, preferably E. coli or Agrobacterium sp. The methods for introducing the expression vector of the present invention to host cells include, but not limited to transformation using Agrobacterium species microorganisms, particle gun bombardment, silicon carbide whiskers, sonication, electroporation and PEG (polyethyleneglycol) precipitation. The method may be preferably the transformation using Agrobacterium species microorganisms or electorporation. Accordingly, the present invention provides a cell transformed with the expression vector of the present invention. The host cell includes eukaryotic cells such as yeast or plant cell, or prokaryotic cells such as E. coli. Beside, the present invention provides plants to which the expression vector was introduced and seeds obtained from the plants.

In the preferred embodiments of the present invention, the inventors constructed pCKAPA recombinant vector by introducing AtKAPAS cDNA into pCAL-n vector (Stratagene, USA) and prepared E. coli transformed with the recombinant vector, which was deposited at Korean Collection for Type Cultures of Korea Research Institute of Biotechnology and Bioscience on Mar. 26, 2002 (Accession No: KCTC 10210BP). Since the above pCAL-n vector contains calmodulin-binding peptide tag sequence, the protein expressed by the vector can be easily separated by calmodulin resin.

The present invention further provides a method for inducing plant growth inhibition by suppressing the expression or function of the polyplkptide involved in biotin synthesis, resulting in inhibition of biotin biosynthesis.

In order to suppress the expression of the said polypeptide, conventional methods generally known to those skilled the pertinent art are all available, that is, antisense polynucleotide introduction, gene deletion, gene insertion, T-DNA introduction, homologous recombination or transposon tagging, etc., and particularly, the method to introduce antisense polynucleotide into plants was used in this invention. The present inventors prepared an antisense construct containing antisense polynucletide sequence of AtKAPAS, a polynucletide having a nucleotide sequence represented by SEQ. ID. NO. 1. Precisely, the inventors constructed pSEN-K recombinant vector by introducing a polynucleotide having a nucleotide sequence represented by SEQ. ID. NO. 1 into pSEN vector in the antisense orientation and transformed E. coli with the prepared recombinant vector. The E. coli comprising the pSEN-K recombinant vector was deposited at Korean Collection for Type Cultures of Korea Research Institute of Biotechnology and Bioscience on Mar. 26, 2002 (Accession No: KCTC 10211BP).

The function of the polypeptide according to the present invention is preferably suppressed by treatment of chemical substances. As a chemical substance used for suppressing the function of the polypeptide, pimeloyl CoA derivatives working as a substrate to KAPA synthase in the biotin biosynthesis pathway can be selected. As stated in an embodiment of the present invention, the polypeptide expressed from the AtKAPAS polynucleotide has strong substrate-specificity especially to pimeloyl CoA. Therefore, pimeloyl CoA derivatives working as competitive inhibitors with pimeloyl CoA can effectively inhibit biotin biosynthesis in plants, and thus, they can be used for inducing plant growth inhibition.

In another embodiment of the present invention, the inventors inhibited protein synthesis from AtKAPAS by transforming Arabidopsis thaliana with the pSEN-K recombinant vector. As a result, it was confirmed that serious retardation of plant growth, leaf etiolation and plant death were induced by the antisense construct of AtKAPAS. Therefore, based on the fact that the antisense construct effectively inhibits plant growth, the present inventors confirmed that the polynucleotide of the present invention could be effectively used for the development of a novel herbicide.

Accordingly, the present invention provides a method for identifying herbicidal compounds that inhibit the expression or function of the polypeptide involved in biotin synthesis. Particularly, the present invention provides a method of identifying herbicidal compounds that inhibit the expression or function of the polypeptide of claim 1, comprising the steps of;

(a) combining a polypeptide of claim 1 with the compounds to be tested for the ability to inhibit the expression or the function of the polypeptide under conditions conducive to inhibition;

(b) selecting the identified compounds to inhibit the expression or function of the polypeptide in step (a);

(c) applying the said compounds selected in step (b) to a plant to test for herbicidal activity; and

(d) selecting the identified compounds to have herbicidal activity in step (c).

The term “herbicidal compounds” refer to substances used to kill or suppress the growth of plants, plant cells, plant seeds, or plant tissues. The effect of herbicidal compounds on expression or function of the polypeptide according to the present invention can be identified by northern blot analysis and western blot analysis that are known to those skilled in the pertinent art. The herbicidal compounds may be, for example, peptides, polypeptides, peptide imitators, chemical compounds, biologicals and the like.

### BRIEF DESCRIPTION OF THE DRAWINGS

The application of the preferred embodiments of the present invention is best understood with reference to the accompanying drawings, wherein:

FIG. 1 is a diagram showing the putative amino acid sequence encoded by the nucleotide sequence of Arabidopsis thaliana AtKAPAS gene, compared with KAPA synthase amino acid sequences of Escherichia coli, Bacillus subtilis and Bacillus sphaericus. Marked similar amino acid sequence regions with black blocks;

FIG. 2 is a graph showing the protein content in each fraction purified by calmodulin affinity chromatography using 2 mM EDTA from crude extracts purified from E. coli transformed with recombinant vector containing AtKAPAS gene and control E. coli transformed with a vector only;

[symbol] : E. coli transformed with recombinant vector containing AtKAPAS gene,

[symbol] : Control E. coli transformed with a vector only

FIG. 3 is a photograph showing the result of SDS-PAGE electrophoresis on fractions #12-14 among every chromatography fraction obtained from effluents of both E. coli transformed with recombinant vector containing AtKAPAS gene and control E. coli;

Lane 1: Fraction #12 of effluents obtained from E. coli transformed with recombinant vector containing AtKAPAS gene,

Lane 2: Fraction #12 of effluents obtained from control E. coli,

Lane 3: Fraction #13 of effluents obtained from E. coli transformed with recombinant vector containing AtKAPAS gene,

Lane 4: Fraction #13 of effluents obtained from control E. coli,

Lane 5: Fraction #14 of effluents obtained from E. coli transformed with recombinant vector containing AtKAPAS gene,

Lane 6: Fraction #14 of effluents obtained from control E. coli

FIG. 4 is a graph showing the Lineweaver-Burk plot reflecting the changes of reaction speed according to the changes of substrate concentration, which was available for the measurement of the substrate-specific enzyme activity of the purified protein to pimeloyl CoA;

FIG. 5 is a diagram showing the structure of a vector into which AtKAPAS gene was introduced in the antisense orientation;

FIG. 6A is a photograph showing saplings growing up from seeds of transgenic Arabidopsis thaliana to which AtKAPAS gene was introduced in the antisense orientation (the saplings were grown and differentiated in a pot with biotin);

FIG. 6B is a photograph showing saplings growing up from seeds of transgenic Arabidopsis thaliana to which AtKAPAS gene was introduced in the antisense orientation (the saplings were grown and differentiated in a pot without biotin).

### BEST MODE FOR CARRYING OUT THE INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Isolation of E. coli bioF Homologous Gene from Arabidopsis thaliana

The present inventors performed screening to isolate homologous genes corresponding to E. coli bioF, a gene encoding KAPA synthase of biotin biosynthesis pathway, from Arabidopsis thaliana.

<1-1> Arabidopsis thaliana Cultivation and Culture

The present inventors cultured Arabidopsis thaliana either in pots filled with soil or in petri dishes containing MS (Murashige and Skoog salts, Sigma, USA) medium supplemented with 2% sucrose (pH 5.7) and 0.8% agar. Put the pots in growth chambers where the temperature was kept at 22° C. and the light and dark cycle was set to 16/8 hours.

<1-2> RNA Extraction and cDNA Library Preparation

In order to construct Arabidopsis thaliana cDNA library, the total RNA was extracted from Arabidopsis thaliana leaves in various differentiation stages using TRI reagent (Sigma, USA). Poly(A)+ RNA was isolated from the extracted total RNA according to the protocol of mRNA isolation kit (Pharmacia, USA). Double stranded cDNA was prepared with poly(A)+ RNA and cDNA synthesis kit (Time Saver cDNA synthesis kit, Pharmacia, USA) using NotI-(dT)18 primer.

<1-3> Isolation of bioF Homologous Gene

Based on the nucleotide sequence of E. coli bioF gene, the present inventors synthesized a forward primer (SEQ. ID. NO. 3) including the sequence of restriction enzyme BamHI and a reverse primer (SEQ. ID. NO. 4) including the sequence of restriction enzyme of HindIII. By performing PCR (polymerase chain reaction) with the two primers, the full-length cDNA was amplified and isolated from Arabidopsis thaliana cDNA library prepared in the above Example <1-2>.

From the sequence analysis of the isolated cDNA, it was confirmed that the cDNA has about 51.3 kDa molecular weight and contains 1410 bp size open reading frame encoding 469 amino acids, and the cDNA was named AtKAPAS (Arabidopsis thaliana KAPA synthase).

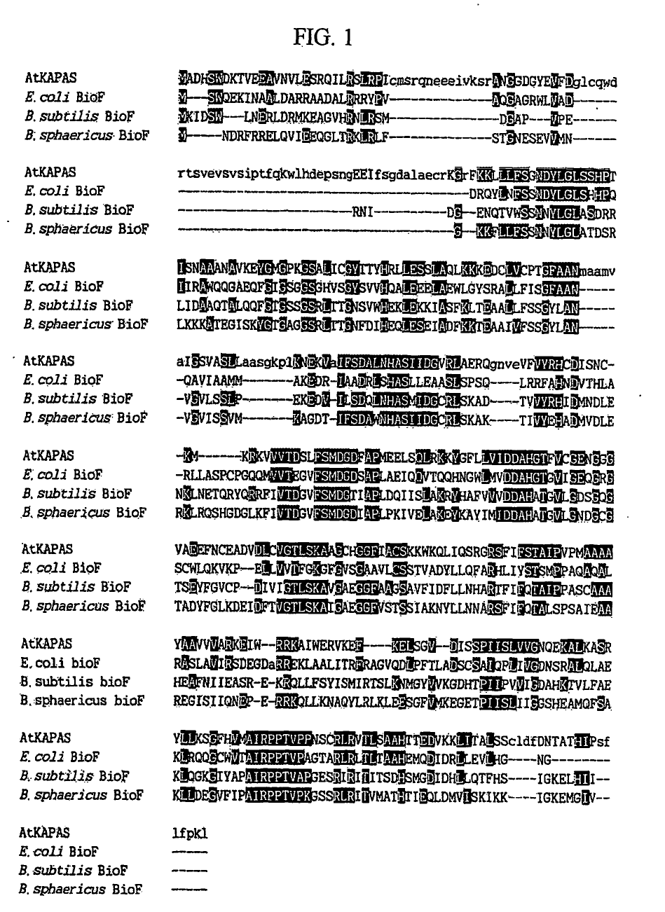

The putative amino acid sequence encoded by the AtKAPAS showed 28%, 34% and 38% homology and 43%, 52% and 54% similarity to the sequences of bioF genes (GeneBank accession number NP286539, NP390900 and JQ0512, each) of Escherichia coli, Bacillus subtilis and Bacillus sphaericus (see FIG. 1), respectively.

Besides, it was confirmed that the above protein contains aminotransferase class I and class II domain at C-terminal region, and a domain predicted to be the plasma membrane spanning region.

### EXAMPLE 2

### Purification of Protein Expressed by AtKAPAS Gene in E. coli

<2-1> Induction of Protein Expression

The amplified DNA fragment comprising the full-length AtKAPAS cDNA isolated in the Example <1-3> was cut with restriction enzymes of BamHI and Hind III, and then constructed pCKAPA recombinant vector by cloning the fragment in the region of BamHI and HindIII site of pCAL-n vector (Stratagene, USA).

E. coli BL21-Gold(DE)(Stratagene, USA) was transformed with the pCKAPA recombinant vector (Accession No: KCTC 10210BP), and then cultured in LB (Luria-Bertani broth, USB, USA) medium containing 100 μg/ml of ampicillin at 37° C. with agitation (150 rpm) until the value of OD₆₀₀ reached 0.7. In order to induce the expression of the target protein in E. coli cells, IPTG (isopropyl-D-thiogalactoside) was added to the suspension at a final concentration of 1 mM, and further cultured for 2 more hours. The culture cells were washed with 50 mM potassium phosphate buffer (pH 7.0) containing 50 mM MgSO₄ and 0.4 M NaCl, after which centrifuged at 4,000×g for 15 minutes. The precipitates thereof were collected and stored at −20° C.

<2-2> Protein Purification

The cell precipitates obtained in the above Example <2-1> were suspended in CaCl₂ binding buffer (50 mM Tris-HCl, pH 8.0, 150 mM NaCl, 10 mM β-mercaptoethanol, 1.0 mM magnesium acetate, 1.0 mM imidazole, 2 mM CaCl₂). Lysozyme was added into the cell suspension at a final concentration of 200 μg/ml and the cell suspension was stirred for 15 minutes. Then, ultrasonication was performed for 30 seconds. The crushed samples were cooled down with ice for 5 minutes. Such procedure (cooling after ultrasonication) was repeated three times. The samples were centrifuged at 10,000×g for 5 minutes to obtain supernatants. The supernatants were purified by calmodulin affinity chromatography. Precisely, the supernatants (crude extracts) were applied onto the equilibrated caanodulin affinity chromatography resin, followed by the reaction at 4° C. for 24 hours. In order to remove unattached proteins and other substances, the column was washed with CaCl₂ binding buffer. The proteins attached to the calmodulin were isolated from column matrix using elution buffer (50 mM Tris-HCl, pH 8.0, 10 mM β-mercaptoethanol, 2 mM EDTA, 150 mM NaCl). The proteins isolated from crude extracts of E. coli transformed with pCAL-n vector were used as a control group.

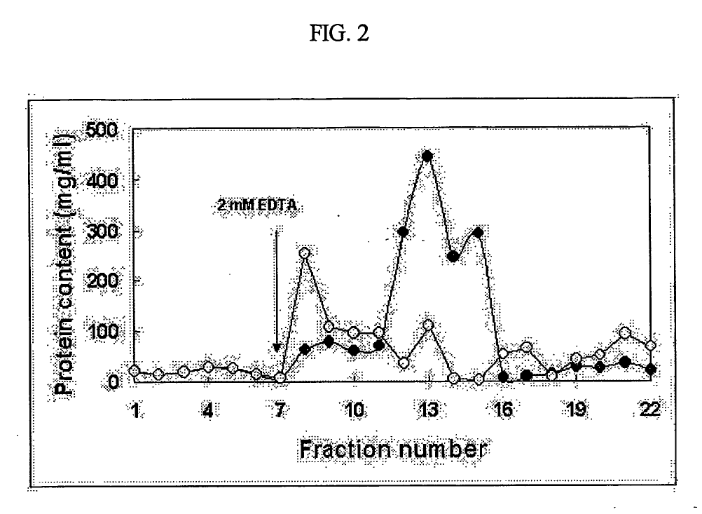

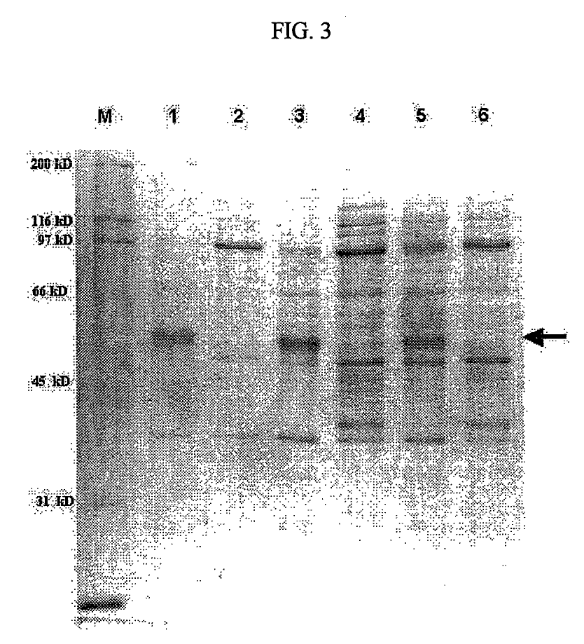

As a result, as seen in FIG. 2, fractions #12-14 of all the fractions separated from E. coli transformed with pCKAPA recombinant vector showed the highest protein content. In order to make sure the purity of the proteins, the fractions #12-14 selected from all effluents separated from E. coli transformed with pCKAPA recombinant vector and the control group E. coli, respectively, were subjected to SDS-PAGE. As a result, as seen in FIG. 3, it was confirmed that the effluents separated from E. coli transformed with pCKAPA recombinant vector contain a 55 kDa size fusion protein (51.3 kDa (molecular weight of the protein expressed by AtKAPAS gene)+4 kDa (molecular weight of calmodulin binding peptide)). On the other hand, it was confirmed that the effluents separated from the control group E. coli do not contain such size protein.

### EXAMPLE 3

### Analysis on the Enzyme Activity of the Protein

In order to investigate whether the isolated protein has the function of KAPA syntase that relates to biotin biosynthesis, the present inventors measured the enzyme activity using pimeloyl CoA as a substrate.

The enzyme activity of the protein was measured at 340 nm using a spectrophotometer (Backman DU series 60 spectrophotometer) adjusted at 30° C. according to a method of Alexeev et al.(Alexeev et al. J. Mol. Biol., 284:401-419, 1998). In the 1 ml of reaction solution for the measurement of the enzyme activity, 20 mM potassium phosphate (pH 7.5), 1 mM NAD+, 3 MM MgCl₂, 0.1 unit α-ketoglutarate dehydrogenase and 2-10 μg protein purified in the above Example <2-2> were included. In every reaction solution, the concentration of the protein was 10 μM. L-alanine and pimeroyl CoA were also added into each reaction solution (0-30×10⁸ M⁻¹). Results were analyzed using the Soft-Pac Module kinetics software. Before the analysis, enzyme samples were dialyzed with 20 mM potassium phosphate buffer (pH 7.5) containing 100 μM PLP (pyridoxal 5′-phosphate) at 4° C. for 2 hours. The cuvette containing other substances but the purified protein was used as a control group for the measurement of the enzyme activity.

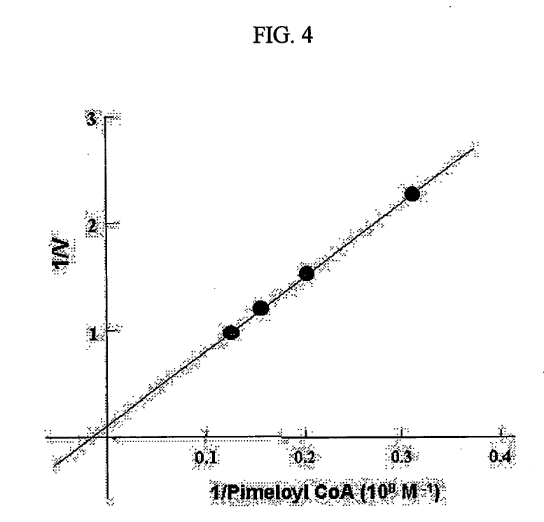

The enzyme activity of the purified protein was suited to Michaelis-Menten kinetics for pimeroyl CoA, a substrate. The changes of reaction speed according to substrate concentration were measured, and then represented by Lineweaver-Burkes plot (see FIG. 4). As a result, Km value and Vmax value were 5.4×10⁻⁷M and 7.93, respectively. Such results supported the fact that the protein expressed by AtKAPAS gene is KAPA syntase having substrate specificity to pimeroyl CoA. Also, the Km values of KAPA syntases of E. coli and Bacillus sphaericus to pimeroyl CoA were 0.5 mM and 2.5 mM each, while Km value of the protein of the present invention was 5.4×10⁻⁷M. Thus, the protein of the present invention was confirmed to have high substrate specificity to pimeroyl CoA, compared with those of E. coli and Bacillus.

### EXAMPLE 4

### Preparation Transgenic Arabidopsis thaliana in Which Antisense Construct of AtKAPAS Gene was Introduced, and Its Characteristic analysis

<4-1> Preparation of Transgenic Arabidopsis thaliana in Which Antisense Construct of AtKAPAS Gene was Introduced

In order to investigate physiological characteristics of the protein purified in the above Example <2-2>, the present inventors prepared transgenic Arabidopsis thaliana in which AtKAPAS gene was introduced in the antisense orientation, resulting in the inhibition of the expression of AtKAPAS transcript.

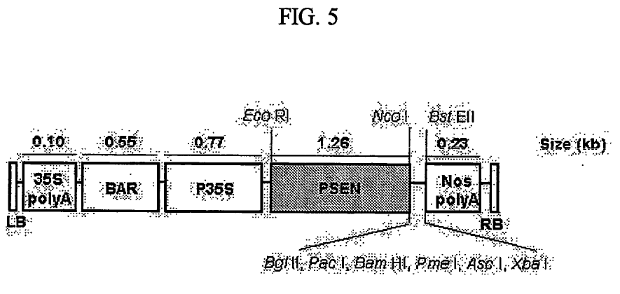

The AtKAPAS DNA was amplified from cDNA of Arabidopsis thaliana by PCR using primers including BglII site and represented by SEQ. ID. NO. 5 and 6, respectively. The amplified DNA was cut with restriction enzyme BglII and cloned into pSEN vector prepared to be regulated not by CaMV 35S promoter of pCAMBIA-3301 vector (provided by a laboratory, Department of Life Science, Pohang University of Science and Technology, Korea) but by senl promoter in the antisense orientation in order to avoid the plant death in germination stage, leading to the construction of pSEN-K recombinant vector (see FIG. 5). The senl promoter has specificity to genes expressed in each phase of plant growth.

The pSEN-K recombinant vector was introduced into Agrobacterium tumefaciens using electroporation. The transformed Agrobacterium tumefaciens was cultured at 28° C. until the OD₆₀₀ reached 1.0. The cells were collected by centrifugation at 5,000 rpm for 10 minutes at 25° C. The collected cells were suspended in IM (Infiltration Medium; 1× MS SALTS, 1× B5 vitamin, 5% sucrose, 0.005% Silwet L-77, Lehle Seed, USA) medium until OD₆₀₀ reached 2.0. The 4-week-old Arabidopsis thaliana was dipped in Agrobacterium tumefaciens suspension in a vacuum chamber and stored under vacuum condition (10⁴ Pa) for 10 minutes. After dipping, Arabidopsis thaliana was put in with polyethylene foil for 24 hours. The transformed Arabidopsis thaliana was grown to obtain seeds (T1). For the control groups, wild type Arabidopsis thaliana that was not transformed and Arabidopsis thaliana transformed with pSEN vector without antisense AtKAPAS gene were selected and used.

<4-2> Analysis on the Characteristics of Transgenic Arabidopsis thaliana T1

Seeds harvested from the transgenic Arabidopsis thaliana obtained in Example <4-1> were selected by dipping it in 0.1% Basta (Kyungnong Co., Korea) solution for 30 minutes and culturing. The transgenic Arabidopsis thaliana showed retarded growth or plant death, compared with those of control group (Arabidopsis thaliana transformed with pSEN vector).

In order to confirm whether the transgenic plant transformed with antisense construct of AtKAPAS gene is a biotin auxotrophic mutant, the reactions induced by adding biotin was investigated. Particularly, 5000 transformed seeds, which were dipped in Basta solution for 30 minutes, were cultured in petri-dishes containing MS medium with or without 5 μM of biotin. And also, the transformed seeds were cultured in pots filled with sands containing 1 mM of biotin or not. The pots were treated with Basta 5 times, and then investigated for the growth of Arabidopsis thaliana in every pot.

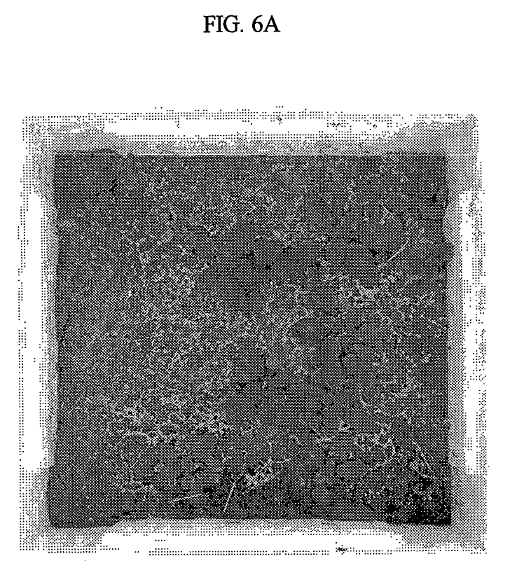

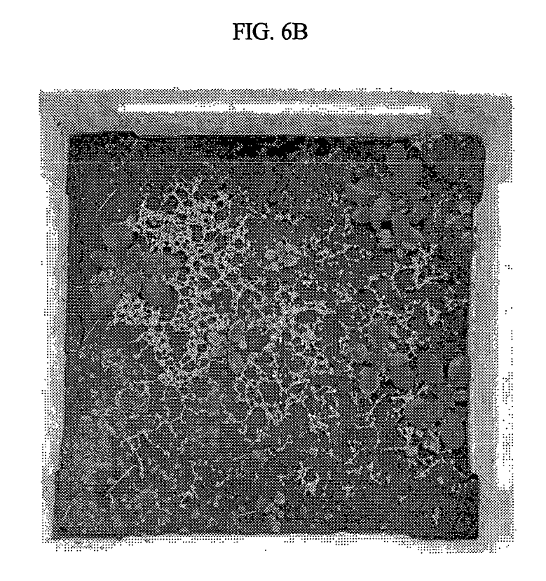

As a result, 16 individual plants were grown up in biotin added pots and their phenotype was not much different from that of wild type Arabidopsis thaliana. Meanwhile, just 11 individual plants were grown up in pots without biotin. 4 of those showed retarded growth, 2 died and their leaves turned yellow (see FIGS. 6A and 6B). Therefore, the transgenic plants comprising the antisense construct of AtKAPAS gene were proved to be biotin auxotrophic mutants.

### INDUSTRIAL APPLICABILITY

The present invention provides a polypeptide involved in biotin biosynthesis in plants, a polynucleotide encoding the said polypeptide, a method for inducing plant growth inhibition by suppressing the expression or function of the said polypeptide, resulting in inhibition of biotin biosynthesis, and a method for identifying herbicidal compounds that inhibit the expression or function of the said polypeptide. Since biotin biosynthesis pathway is present neither in human nor in animals, the herbicidal compounds having functions to suppress the expression or function of the polypeptide participating in biotin biosynthesis have an advantage of doing no harm to human and animals. Therefore, such herbicidal compounds can be effectively used as environmental friendly and safe herbicides.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An isolated polypeptide having an amino acid sequence selected from the group consisting of;
(a) an amino acid sequence of SEQ. ID. NO. 2, and
(b) an amino acid sequence having at least 70% identity to SEQ. ID. NO. 2.

2. The polypeptide of claim 1, wherein the polypeptide having the amino acid sequence of SEQ. ID. NO. 2 contains aminotransferase class I and II domain at C-terminal.

3. An isolated polynucleotide encoding the polypeptide of claim 1.

4. The polynucleotide of claim 3, wherein the polynucleotide comprises a nucleotide sequence of SEQ. ID. NO. 1.

5. An expression vector comprising the polynucleotide of claim 3.

6. The expression vector of claim 5, wherein the expression vector comprises the polynucleotide having a nucleotide sequence of SEQ. ID. NO. 1.

7. A cell comprising the expression vector of claim 5.

8. A transgenic plant comprising the expression vector of claim 5.

9. A seed comprising the expression vector of claim 5.

10. An antisense polynucleotide complementary to the polynucleotide of claim 3.

11. The antisense polynucleotide of claim 10, wherein the antisense polynucleotide is complementary to the polynucleotide having a nucleotide sequence of SEQ. ID. NO. 1.

12. An expression vector comprising the antisense polynucleotide of claim 10.

13. The expression vector of claim 12, wherein the expression vector comprises an antisense polynucleotide, wherein the antisense polynucleotide is complementary to the polynucleotide having a nucleotide sequence of SEQ. ID. NO. 1.

14. A cell comprising the expression vector of claim 12.

15. A transgenic plant comprising the expression vector of claim 12.

16. A seed comprising the expression vector of claim 12.

17. A method for inducing plant growth inhibition by suppressing the expression or function of the polypeptide of claim 1, resulting in inhibition of biotin biosynthesis.

18. The method of claim 17, wherein the suppression of the expression of the polypeptide is achieved by any method selected from the group consisting of introduction of an antisense polynucleotide, gene deletion, gene insertion, T-DNA introduction, homologous recombination and transposon tagging, wherein the antisense polynucleotide is complementary to an isolated polynucleotide encoding a polypeptide, and wherein the polypeptide has an amino acid sequence selected from the group consisting of:
(a) an amino acid sequence of SEQ. ID. NO. 2; and
(b) an amino acid sequence having at least 70% identity to SEQ. ID. NO. 2.

19. A method for identifying herbicidal compounds that inhibit the expression or function of the polypeptide of claim 1, comprising the steps of;
(a) combining a polypeptide of claim 1 with the compounds to be tested for the ability to inhibit the expression or the function of the polypeptide under conditions conducive to inhibition;
(b) selecting the identified compounds to inhibit the expression or function of the polypeptide in step (a);
(c) applying the said compounds selected in step (b) to a plant to test for herbicidal activity; and
(d) selecting the identified compounds to have herbicidal activity in step (c).


SEQUENCE LISTING (6 sequences)

SEQ ID NO: 1 - DNA, 1410 bp, Arabidopsis thaliana
atggcggatcattcgtgggataaaactgtggaagaagcagtgaatgtgcttgaatccaggcaaattcttcgatctttgaggcccatttgcatgtctaggcaaaacgaagaagaaatagtgaaaagcagagccaatggaggagacgggtacgaggtgttcgacggtttgtgtcaatgggatcggacttcagttgaggtgtctgtctcgattcctacatttcagaaatggcttcacgatgaacccagcaacggagaagagatttttagtggagatgcattagctgagtgtagaaaagggagattcaagaagctgcttttgttctctgggaatgattatttgggtttgagctcacatcctacaatatcaaacgctgctgcaaacgcagtcaaagaatatggtatgggacctaagggttctgctttaatatgtggctataccacttatcatcgtttgcttgagtctagtttggcgcaactgaagaaaaaagaggattgtcttgtttgtcctactgggtttgctgccaatatggctgcaatggttgcaattggaagtgttgcttctcttttggccgctagcgggaaacctctgaagaatgaaaaagttgccatcttttctgatgcgctgaatcatgcatcaattattgatggtgtccgtcttgctgaacgacaaggaaatgttgaagtttttgtttatcgacactgtgacatatcaaattgcaaaatgaagaggaaggtcgtggtgactgatagcttatttagtatggacggtgactttgcaccaatggaagagctctctcagcttcggaagaagtatggcttccttctagttattgatgatgctcatggaacatttgtctgtggagaaaacggtggtggcgtggctgaggaatttaactgtgaagctgatgtagatttatgtgtgggcactttgagtaaggcagcagggtgtcatggcggtttcatagcttgcagcaaaaaatggaagcaactgatacagtcgagaggtcgttcattcatattttcaacagcaatccctgtcccaatggctgcagctgcttatgcagcagttgtagtggcgaggaaggagatatggagaagaaaggcaatatgggagagggtaaaagagttcaaggaattatctggagttgacatctcaagccccattatctcacttgttgtagggaatcaagagaaagccctcaaagcgagccggtatctattaaaatcaggcttccatgtaatggcaatacgaccgcccacagtgccacccaattcttgcaggctaagggtgacactgagtgcagcacataccacagaagatgtgaagaaactcatcactgcgctttcttcttgtttggactttgacaacacagccactcacattccttcctttctatttcccaaattataa

SEQ ID NO: 2 - protein, 469 aa, Arabidopsis thaliana
MADHSWDKTVEEAVNVLESRQILRSLRPICMSRQNEEEIVKSRANGGDGYEVFDGLCQWDRTSVEVSVSIPTFQKWLHDEPSNGEEIFSGDALAECRKGRFKKLLLFSGNDYLGLSSHPTISNAAANAVKEYGMGPKGSALICGYTTYHRLLESSLAQLKKKEDCLVCPTGFAANMAAMVAIGSVASLLAASGKPLKNEKVAIFSDALNHASIIDGVRLAERQGNVEVFVYRHCDISNCKMKRKVVVTDSLFSMDGDFAPMEELSQLRKKYGFLLVIDDAHGTFVCGENGGGVAEEFNCEADVDLCVGTLSKAAGCHGGFIACSKKWKQLIQSRGRSFIFSTAIPVPMAAAAYAAVVVARKEIWRRKAIWERVKEFKELSGVDISSPIISLVVGNQEKALKASRYLLKSGFHVMAIRPPTVPPNSCRLRVTLSAAHTTEDVKKLITALSSCLDFDNTATHIPSFLFPKL

SEQ ID NO: 3 - DNA, 28 bp, Artificial Sequence
  Forward primer for AtKAPAS gene
ggcggatccttcgcccaaatcacaattc

SEQ ID NO: 4 - DNA, 32 bp, Artificial Sequence
  Reverse primer for AtKAPAS gene
ggcaagcttttcactgacaatatcagaaacaa

SEQ ID NO: 5 - DNA, 26 bp, Artificial Sequence
  Forward primer for AtKAPAS gene
gcagatcttcgcccaaatcacaattc

SEQ ID NO: 6 - DNA, 31 bp, Artificial Sequence
  Reverse primer for AtKAPAS gene
gcagatctttcactgacaatatcagaaacaa